(19) United States
(12) Patent Application Publication
(10) Pub. No.: US 2021/0330291 A1
(43) Pub. Date: Oct. 28, 2021

(54) ULTRASONIC PROBE AND ULTRASONIC APPARATUS

(71) Applicant: General Electric Company, Schenectady, NY (US)

(72) Inventors: Yasuo Yoshikawa, Tokyo (JP); Mitsuhiro Nozaki, Tokyo (JP)

(21) Appl. No.: 17/239,920

(22) Filed: Apr. 26, 2021

(30) Foreign Application Priority Data
Apr. 28, 2020 (JP) 2020-078844

(51) Int. Cl. A61B 8/00

(52) U.S. Cl. CPC A61B 8/4444; A61B 8/4494

(57) ABSTRACT

Various ultrasonic probes and ultrasonic apparatuses are provided in which the size of an area of contact with a patient is reduced while maintaining the strength of a housing of the ultrasonic probe. In an exemplary aspect, the ultrasonic probe includes a housing for housing an array of transducer elements and an acoustic lens disposed in an end portion of said housing. The housing includes a first portion in which an acoustic lens is disposed and a second portion. The first portion is constructed, at least in part, from a first material having a Young's modulus of 40 GPa or higher and also having electrical insulation.

### FIELD

The present invention relates to an ultrasonic probe and an ultrasonic apparatus including the ultrasonic probe in which a size of an area of contact of the ultrasonic probe with a patient is reduced while maintaining the strength of the ultrasonic probe.

### BACKGROUND

Ultrasonic probes have a housing containing an array of transducer elements. The housing in conventional ultrasonic probes is constructed from a thermoplastic resin, for example. The housing is provided in its end portion with an acoustic lens. An ultrasonic scan is performed while putting a lens surface of the acoustic lens against a patient.

In many applications, ultrasonic energy is transmitted/received in a limited space, such as the intercostal regions of a human body, and an ultrasonic probe having a reduced size of an area of contact with a patient is employed. The size of the contact area is based on the lens surface of the acoustic lens and the thickness of an end portion of the housing in which the acoustic lens is disposed. Therefore, to reduce the size of the contact area, it is desirable to reduce the thickness of the end portion of the housing as much as possible.

As the thickness of the housing constructed from a thermoplastic resin is reduced, however, the housing is more vulnerable to strain and/or breakage from an external impact. This potentially leads to destruction of an internal structure, such as the transducer element array, by transmission of the impact to the inside.

Moreover, the inside of the housing containing the array of ultrasonic transducers, needs to be electrically insulated from the outside of the housing. For at least these reasons, there is a need for an improved ultrasonic probe and an improved ultrasonic apparatus including the improved ultrasonic probe.

### BRIEF SUMMARY

In an embodiment, an ultrasonic probe includes a housing for housing an array of transducer elements and an acoustic lens disposed in an end portion of the housing. The end portion is constructed, at least in part, from a first material having a Young's modulus of 40 GPa or higher and also having electrical insulation.

In an embodiment, an ultrasonic apparatus includes an ultrasonic probe. The ultrasonic probe includes a housing for housing an array of transducer elements and an acoustic lens disposed in an end portion of the housing. The end portion of the housing is constructed, at least in part, from a first material having a Young's modulus of 40 GPa or higher and also having electrical insulation.

### BRIEF DESCRIPTION OF THE DRAWINGS

FIG. 1 is a cross-sectional view showing part of an ultrasonic probe in accordance with an embodiment;

FIG. 2 is a perspective view showing a first portion included in the ultrasonic probe in FIG. 1;

FIG. 3 is a diagram explaining a thickness of the first portion;

FIG. 4 is a block diagram showing an example of an ultrasonic diagnostic apparatus in accordance with an embodiment;

FIG. 5 is a cross-sectional view showing part of an ultrasonic probe in accordance with a variation of the embodiment; and

FIG. 6 is a perspective view showing a second portion and a second portion included in the first portion in accordance with an embodiment.

### DETAILED DESCRIPTION

Embodiments of the present disclosure will now be described, by way of example, with reference to the Figures. The ultrasonic probe in the embodiments may be any one of a linear probe, a sector probe, and a convex probe, or any other type of ultrasonic probe. The following embodiments will address a sector probe in accordance with a non-limiting example.

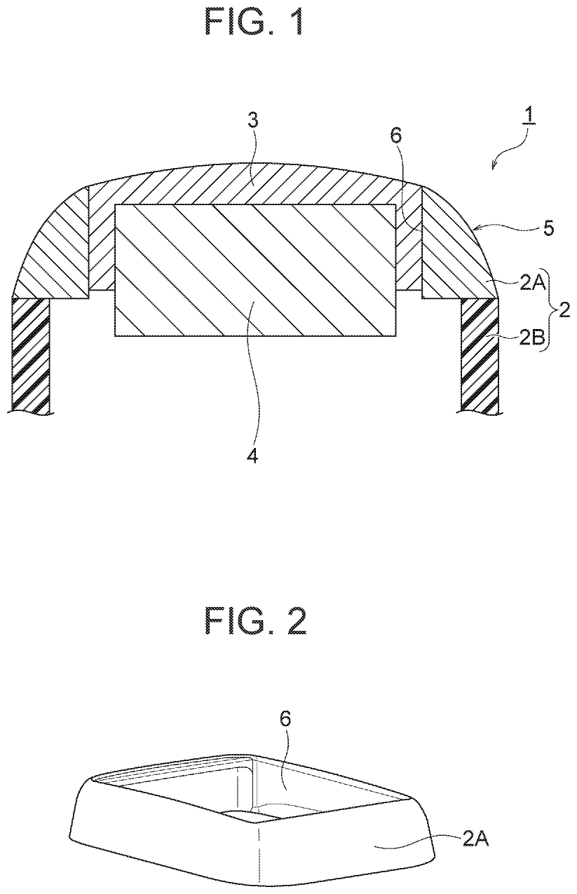

FIG. 1 is a cross-sectional view showing part of an ultrasonic probe 1 in accordance with an embodiment. The ultrasonic probe 1 performs an ultrasonic scan on a patient, and receives echo signals. The ultrasonic probe 1 includes a housing 2, an acoustic lens 3, and an acoustic element module 4. An end portion 5 of the housing 2 defines an opening portion 6, where the acoustic lens 3 is disposed. The acoustic element module 4 is contained in the housing 2 in contact with the acoustic lens 3. Although other components known in the art are contained in the housing 2, they are not shown in FIG. 1.

The acoustic element module 4 has a configuration known in the art, which is not shown in detail, comprising a transducer element array 7 (see FIG. 4), an acoustic matching layer, a backing, etc. The transducer element array 7 emits pulsed ultrasound to a patient (not shown). More specifically, the pulsed ultrasound from the transducer element array 7 passes through the acoustic lens 3, and penetrates into the patient. The pulsed ultrasound penetrating into the patient is reflected from structures inside of the patient to generate echoes. The echoes from the patient pass through the acoustic lens 3 to reach the transducer element array 7. The transducer element array 7 converts the echoes for the pulsed ultrasound into electrical signals.

The housing 2 includes a first housing portion 2A constituting the end portion 5, as shown in FIG. 2, and further includes a second housing portion 2B. The first housing portion 2A covers at least part of the acoustic element module 4, and therefore, it also covers at least part of the transducer element array 7 included in the acoustic element module 4.

The first housing portion 2A and second housing portion 2B are coupled to each other by a technique known in the art. The structure of coupling is omitted in the drawings. The housing is constructed so that liquid is prevented from penetrating into the inside of the housing 2 from between the first housing portion 2A and second housing portion 2B.

The first housing portion 2A is constructed from a first material. The first material has a degree of elasticity (Young's modulus) of 40 GPa or higher, and also has electrical insulation. In an example, the term ‘electrical insulation’ means an electrical insulation of 7.5 kV/mm or higher. For example, the first material is a ceramic. The ceramic may be a fine ceramic. Alternatively, the first material may be a metal having a Young's modulus of 40 GPa or higher and also having electrical insulation.

The second housing portion 2B is constructed from a material known in the art as a material for an ultrasonic probe housing. According to an exemplary embodiment, the second housing portion 2B may be constructed from a thermoplastic resin.

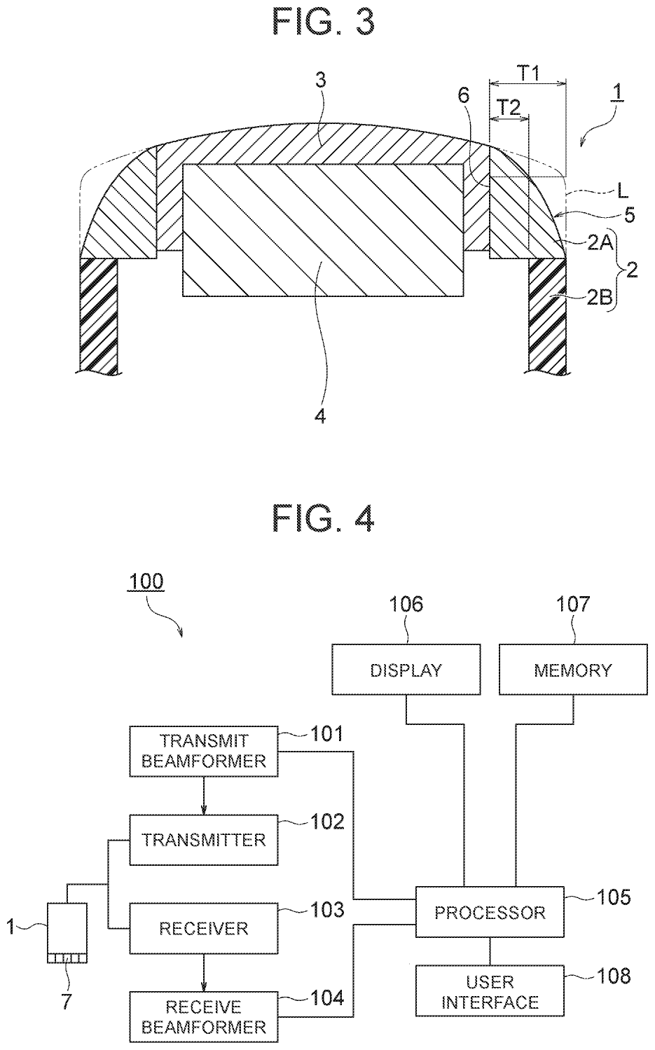

Since the first material has a Young's modulus of 40 GPa or higher as described above, the first housing portion 2A is harder than a conventional housing of thermoplastic resin, so that the strength can be maintained. This makes it possible to reduce the thickness of the first housing portion 2A as compared with the housing of thermoplastic resin. This will be described with reference to FIG. 3. In FIG. 3, a two-dot-dash line L represents part of an outer surface of a hypothetical housing constructed from a thermoplastic resin. T1 designates the thickness of the hypothetical housing, while T2 designates the thickness of the first housing portion 2A as compared with the thickness T1 of the hypothetical housing. Since the first housing portion 2A is constructed from the first material harder than a thermoplastic resin, the thickness T2 can be reduced relative to the thickness T1 of the hypothetical housing while maintaining the strength. This implies that the area of contact with a patient can be reduced.

Moreover, electrical insulation between the inside and outside of the housing 2 can be maintained as well while maintaining the strength as described above.

Next, an ultrasonic apparatus having the ultrasonic probe 1 will be described. The ultrasonic apparatus displays an ultrasonic image produced based on ultrasonic echoes acquired by the ultrasonic probe 1. An ultrasonic diagnostic apparatus 100, which is an example of the ultrasonic apparatus, will be described with reference to FIG. 4.

The ultrasonic diagnostic apparatus 100 shown in FIG. 4 comprises the ultrasonic probe 1, a transmit beamformer 101, and a transmitter 102. The transmit beamformer 101 and transmitter 102 drive the transducer element array 7 to emit pulsed ultrasound.

The ultrasonic diagnostic apparatus 100 further comprises a receiver 103 and a receive beamformer 104. Echoes of the pulsed ultrasound emitted from the transducer element array 7 are converted into electrical signals by the transducer element array 7, which are echo signals, and are input to the receiver 103. The echo signals undergo amplification, etc. with a required gain at the receiver 103, and then input to the receive beamformer 104, where receive beamforming is performed. The receive beamformer 104 outputs receive-beamformed ultrasound data.

The receive beamformer 104 may be a hardware beamformer or a software beamformer. In the case that the receive beamformer 104 is a software beamformer, it may comprise one or more processors including a graphics processing unit (GPU), a microprocessor, a central processing unit (CPU), a digital signal processor (DSP), or any one or more of other kinds of processors capable of executing logical operations. For embodiments where the receive beamformer 104 is a software beamformer, the processor(s) constituting the receive beamformer 104 may be constructed from a processor separate from a processor 105, which will be described later, or constructed from the processor 105.

The ultrasonic probe 1 may include electrical circuitry to perform all or part of the transmit and/or receive beamforming. For example, all or part of the transmit beamformer 101, transmitter 102, receiver 103, and receive beamformer 104 may be situated within the ultrasonic probe 1.

The ultrasonic diagnostic apparatus 100 also comprises the processor 105 for controlling the transmit beamformer 101, transmitter 102, receiver 103, and receive beamformer 104. Moreover, the ultrasonic diagnostic apparatus 100 comprises a display 106, memory 107, and a user interface 108.

The processor 105 is in electronic communication with the ultrasonic probe 1. The processor 105 may control the ultrasonic probe 1 to acquire ultrasound data. The processor 105 controls which of the transducer elements are active, and the shape of an ultrasonic beam transmitted from the ultrasonic probe 1. The processor 105 is also in electronic communication with the display 106, and the processor 105 may process the ultrasound data into ultrasonic images for display on the display 106. The phrase “electronic communication” may be defined to include both wired and wireless connections. The processor 105 may include a central processing unit (CPU) according to one embodiment. According to other embodiments, the processor 105 may include other electronic components capable of carrying out processing functions, such as a digital signal processor, a field-programmable gate array (FPGA), a graphics processing unit (GPU), or any other type of processor. According to other embodiments, the processor 105 may include a plurality of electronic components capable of carrying out processing functions. For example, the processor 105 may include two or more electronic components selected from a list of electronic components including: a central processing unit, a digital signal processor, a field-programmable gate array, and a graphics processing unit.

The processor 105 may also include a complex demodulator (not shown) that demodulates RF data. In another embodiment, the demodulation can be carried out earlier in the processing chain.

The processor 105 is adapted to perform one or more processing operations according to a plurality of selectable ultrasonic modalities on the data. The data may be processed in real-time during a scanning session as the echo signals are received. For the purpose of this disclosure, the term “real-time” is defined to include a procedure that is performed without any intentional delay.

The data may be temporarily stored in a buffer (not shown) during ultrasonic scanning, so that they can be processed in a live operation or in an off-line operation not in real-time. In this disclosure, the term “data” may be used in the present disclosure to refer to one or more datasets acquired with an ultrasonic diagnostic apparatus.

The ultrasound data may be processed by other or different mode-related modules by the processor 105 (e.g., B-mode, color Doppler, M-mode, color M-mode, spectral Doppler, elastography, TVI, strain, strain rate, and the like) to form data for ultrasonic images. For example, one or more modules may produce ultrasonic images in B-mode, color Doppler, M-mode, color M-mode, spectral Doppler, elastography, TVI, strain, strain rate, and combinations thereof, and the like. The image beams and/or image frames are stored and timing information indicating a time at which the data was acquired in memory may be recorded. The modules may include, for example, a scan conversion module to perform scan conversion operations to convert the image frames from coordinate beam space to display space coordinates. A video processor module may be provided that reads the image frames from memory and displays the image frames in real-time while a procedure is being carried out on the patient. The video processor module may store the image frames in image memory, from which the ultrasonic images are read and displayed on the display 106.

The ultrasound data before the scan conversion operations will be referred to herein as raw data. The data after the scan conversion operations will be referred to herein as image data.

In the case that the processor 105 includes a plurality of processors, the aforementioned processing tasks to be handled by the processor 105 may be handled by the plurality of processors. For example, a first processor may be utilized to demodulate and decimate the RF signal while a second processor may be used to further process the data prior to displaying an image.

In the case that the receive beamformer 104 is a software beamformer, for example, its processing functions may be carried out by a single processor or by a plurality of processors.

The display 106 may be an LED (Light Emitting Diode) display, an LCD (Liquid Crystal Display), an organic EL (Electro-Luminescence) display, or the like.

The memory 107 is any known data storage medium, and comprises non-transitory storage media and transitory storage media. The non-transitory storage medium is, for example, a non-volatile storage medium such as an HDD (Hard Disk Drive) and ROM (Read Only Memory). The non-transitory storage media may include a portable storage medium such as a CD (Compact Disk) and a DVD (Digital Versatile Disk). Programs executed by the processor 7 are stored in a non-transitory storage medium

The transitory storage medium may be a volatile storage medium such as RAM (Random Access Memory).

The user interface 108 accepts an operator's input. For example, the user interface 108 accepts an input of a command, information, and/or the like, from a user. The user interface 108 is adapted to include a keyboard, hard keys, a trackball, a rotary control, soft keys, and the like. The user interface 108 may include a touch screen that displays soft keys, etc.

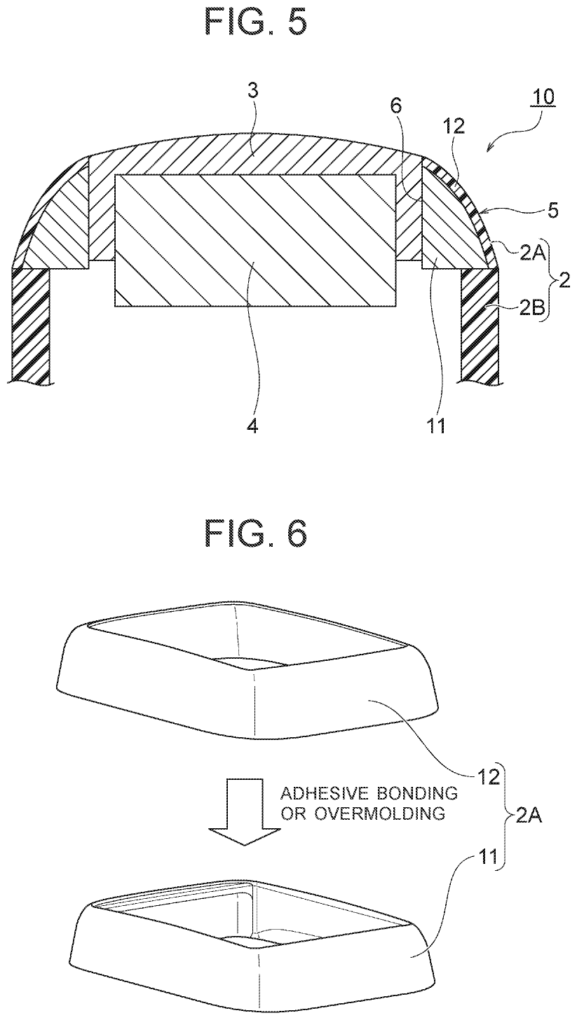

Next, a variation of the embodiment will be described. FIG. 5 is a cross-sectional view showing part of an ultrasonic probe 10 in accordance with a variation of the embodiment. The first housing portion 2A has a first end portion 11 and a second end portion 12. The second end portion 12 is disposed on an outer surface of the first end portion 11. In an example, the first end portion 11 and second end portion 12 are integrally formed by a bonding step or an overmolding step to fabricate the first housing portion 2A, as shown in FIG. 6. The thickness of the second end portion 12 may be in a range of from 0.2 mm to 2 mm, for example.

The first end portion 11 is constructed from the first material described previously. The second end portion 12 is constructed from a second material having a higher elongation than that of the first material. For example, the second material may have an elongation of 2% or higher. The second material is a thermoplastic resin or a silicone rubber, for example. The silicone rubber includes RTV (Room Temperature Vulcanized) silicone.

Since the second end portion 12 disposed on the outer surface of the first end portion 11 has a higher elongation than that of the first end portion 11, the outer surface of the housing 2 is less likely to crack. Moreover, when the second end portion 12, which is the outer surface, is constructed from the thermoplastic resin or silicone rubber, biocompatibility and resistance to chemical solutions and agents can be guaranteed.

The first end portion 11 of the first housing portion 2A provides strength for the housing for embodiments where the second end portion 12 is constructed from a thermoplastic resin since the first end portion 11 is constructed from the first material described hereinabove.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

1. An ultrasonic probe comprising:
a housing for housing an array of transducer elements; and
an acoustic lens disposed in an end portion of said housing, wherein
said end portion is constructed, at least in part, from a first material having a Young's modulus of 40 GPa or higher and also having electrical insulation.

2. The ultrasonic probe as recited in claim 1, wherein said first material is a ceramic or a metal.

3. The ultrasonic probe as recited in claim 1, wherein said first material has an electrical insulation of 7.5 kV/mm or higher.

4. The ultrasonic probe as recited in claim 1, wherein said end portion comprises a first end portion constructed from the first material and a second end portion disposed on an outer surface of said first end portion and constructed from a second material having an elongation higher than that of said first material.

5. The ultrasonic probe as recited in claim 4, wherein said second material has an elongation of 2% or higher.

6. The ultrasonic probe as recited in claim 5, wherein said second material is a thermoplastic resin or a silicone rubber.

7. The ultrasonic probe as recited in claim 1, wherein said housing includes a first housing portion constituting said end portion, and a second housing portion coupled with said first portion.

8. The ultrasonic probe as recited in claim 1, wherein said end portion defines an opening portion in which said acoustic lens is disposed.

9. The ultrasonic probe as recited in claim 1, wherein said end portion is a portion covering said array of transducer elements.

10. An ultrasonic apparatus comprising an ultrasonic probe, the ultrasonic probe comprising:
a housing for housing an array of transducer elements; and
an acoustic lens disposed in an end portion of said housing, wherein
said end portion of said housing is constructed, at least in part, from a first material having a Young's modulus of 40 GPa or higher and also having electrical insulation.

11. The ultrasonic apparatus as recited in claim 10, wherein said first material is a ceramic or a metal having a Young's modulus of 40 GPa or higher and also having electrical insulation.

12. The ultrasonic apparatus as recited in claim 10, wherein said first material has an electrical insulation of 7.5 kV/mm or higher.

13. The ultrasonic apparatus as recited in claim 10, wherein said end portion comprises a first end portion constructed from the first material and a second end portion disposed on an outer surface of the first end portion and constructed from a second material having an elongation higher than that of said first material.

14. The ultrasonic apparatus as recited in claim 13, wherein said second material has an elongation of 2% or higher.

15. The ultrasonic apparatus as recited in claim 14, wherein said second material is a thermoplastic resin or a silicone rubber.

16. The ultrasonic apparatus as recited in claim 10, wherein said housing includes a first housing portion constituting said end portion, and a second housing portion coupled with said first portion.

17. The ultrasonic apparatus as recited in claim 10, wherein said end portion defines an opening portion in which said acoustic lens is disposed.

18. The ultrasonic apparatus as recited in claim 10, wherein said end portion is a portion covering said array of transducer elements.
